(19) United States
(12) Patent Application Publication
(10) Pub. No.: US 2019/0180874 A1
(43) Pub. Date: Jun. 13, 2019

(54) Second Opinion Decision Support Using Patient Electronic Medical Records

(71) Applicant: International Business Machines Corporation, Armonk, NY (US)

(72) Inventors: Uri Kartoun, Cambridge, MA (US); Fang Lu, Billerica, MA (US); Kenney Ng, Arlington, MA (US); Yoonyoung Park, Cambridge, MA (US)

(21) Appl. No.: 16/197,428

(22) Filed: Nov. 21, 2018

Related U.S. Application Data
(63) Continuation of application No. 15/838,610, filed on Dec. 12, 2017.

(51) Int. Cl. G16H 50/20; G06F 16/25; G16H 10/60; G16H 50/70; G16H 70/20; G16H 80/00; G16H 20/10; G06Q 50/00

(52) U.S. Cl. CPC G16H 50/20; G06F 16/25; G16H 10/60; G06Q 50/01; G16H 70/20; G16H 80/00; G16H 20/10; G16H 50/70

(57) ABSTRACT

Mechanisms are provided to implement a second opinion recommendation system that operates to analyze EMRs of a patient to identify a medical condition associated with the patient. The second opinion recommendation system identifies a set of treatments from a corpus of medical treatment guidelines for the patient's medical condition and evaluates the EMRs of the patient to identify a current treatment being performed to treat the patient's medical condition. The second opinion recommendation system compares the current treatment to the set of treatments identified from the corpus of medical treatment guidelines for the patient's medical condition and, responsive to identifying a treatment in the set of treatments corresponding to the current treatment, determines differences between the identified treatment and the current treatment. The second opinion recommendation system sends a notification indicating the determined differences and a recommendation that a second opinion should be sought.

### BACKGROUND

The present application relates generally to an improved data processing apparatus and method and more specifically to mechanisms for evaluating a patient's electronic medical records to identify the send for a second opinion regarding the medical treatment being received by the patient.

Decision-support systems exist in many different industries where human experts require assistance in retrieving and analyzing information. An example that will is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970s uses disease-finding relations and disease-disease relations. The MYCIN system for diagnosing infectious diseases, also developed in the 1970s, uses structured knowledge in the form of production rules, stating that if certain facts are true, then one can conclude certain other facts with a given certainty factor. DXplain, developed starting in the 1980s, uses structured knowledge similar to that of Internist-I, but adds a hierarchical lexicon of findings.

Iliad, developed starting in the 1990s, adds more sophisticated probabilistic reasoning where each disease has an associated a priori probability of the disease (in the population for which Iliad was designed), and a list, of findings along with the fraction of patients with the disease who have the finding (sensitivity), and the fraction of patients without the disease who have the finding (1-specificity).

In 2000, diagnosis systems using unstructured knowledge started to appear. These systems use some structuring of knowledge such as, for example, entities such as findings and disorders being tagged in documents to facilitate retrieval. ISABEL, for example, uses Autonomy information retrieval software and a database of medical textbooks to retrieve appropriate diagnoses given input findings. Autonomy Auminence uses the Autonomy technology to retrieve diagnoses given findings and organizes the diagnoses by body system. First CONSULT allows one to search a large collection of medical books, journals, and guidelines by chief complaints and age group to arrive at possible diagnoses. PEPID DDX is a diagnosis generator based on PEPID's independent clinical content.

Clinical decision rules have been developed for a number of medical disorders, and computer systems have been developed to help practitioners and patients apply these rules. The Acute Cardiac Ischemia Time-Insensitive Predictive Instrument (ACI-TIPI) takes clinical and ECG features as input and produces probability of acute cardiac ischemia as output to assist with triage of patients with chest pain or other symptoms suggestive of acute cardiac ischemia. ACI-TIPI is incorporated into many commercial heart monitors/defibrillators. The CaseWalker system uses a four-item questionnaire to diagnose major depressive disorder. The PKC Advisor provides guidance on 98 patient problems such as abdominal pain and vomiting.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising at least one processor and at least one memory, the at least one memory comprising instructions executed by the at least one processor to cause the at least one processor to implement a second opinion recommendation system. The method comprises analyzing, by the second opinion recommendation system, electronic medical records (EMRs) of a patient to identify a medical condition associated with the patient. The method also comprises identifying, by the second opinion recommendation system, a set of treatments from a corpus of medical treatment guidelines for the patient's medical condition. Moreover, the method comprises evaluating, by the second opinion recommendation system, the EMRs of the patient to identify a current treatment being performed to treat the patient's medical condition. In addition, the method comprises comparing, by the second opinion recommendation system, the current treatment to the set of treatments identified from the corpus of medical treatment guidelines for the patient's medical condition. Further, the method comprises, responsive to identifying a treatment in the set of treatments corresponding to the current treatment, determining, by the second opinion recommendation system, differences between the identified treatment and the current treatment and, sending, by the second opinion recommendation system, a notification indicating the determined differences between the identified treatment and the current treatment and a recommendation that a second opinion should be sought. In this way, the second opinion recommendation system that constantly observes the patient's EMR, including all medications, vitals, lab tests, etc., to determine whether the patient might be at a high risk to be over treated or under treated.

In the illustrative embodiment, the patient being under treated is determined by the patient failing to receive medications or procedures that are recommended by treatment guidelines for patients with similar medical conditions and the patient being over treated is determined by the patient being prescribed medications or procedures that are not recommended by treatment guidelines for patients with similar medical conditions

In some illustrative embodiments, the method further comprises comparing, by the second opinion recommendation system, a recovery process of the patient to a recovery process of other patients that are undergoing a same treatment for the same or similar medical condition; and, responsive to the recovery process of the patient failing to coincide with the recovery process of the other patients that are undergoing the same treatment, sending, by the second opinion recommendation system, a notification indicating that the second opinion should be sought.

In other illustrative embodiments, the method further comprises comparing, by the second opinion recommendation system, a recovery process of the patient to a recovery process of other patients that have undergone a same treatment for the same or similar medical condition and have moved to other treatments for the medical condition; and, responsive to the recovery process of the patient failing to coincide with the recovery process of the other patients that have undergone the same treatment, sending, by the second opinion recommendation system, a notification indicating that the second opinion should be sought along with an indication of the other treatments for the medical condition. In the illustrative embodiment, the other patients are further limited by having a similar demographic profile to that of the patient.

In some illustrative embodiments, the method further comprises recommending, by the second opinion recommendation system, that a medical professional implementing the current treatment be consulted. In some illustrative embodiments, the method further comprises recommending, by the second opinion recommendation system, that a different medical professional other than the medical professional implementing the current treatment be consulted. In the illustrative embodiment, the different medical professional is identified by analyzing social media of the patient.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment. For example, in some illustrative embodiments, a computer program product comprising a computer readable storage medium having a computer readable program stored therein is provided, where the computer readable program, when executed on a computing device, causes the computing device to implement a second opinion recommendation system which operates to analyze electronic medical records (EMRs) of a patient to identify a medical condition associated with the patient; identify a set of treatments from a corpus of medical treatment guidelines for the patient's medical condition; evaluate the EMRs of the patient to identify a current treatment being performed to treat the patient's medical condition; compare the current treatment to the set of treatments identified from the corpus of medical treatment guidelines for the patient's medical condition; responsive to identifying a treatment in the set of treatments corresponding to the current treatment, determine differences between the identified treatment and the current treatment; and send a notification indicating the determined differences between the identified treatment and the current treatment and a recommendation that a second opinion should be sought.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment. For example, in some illustrative embodiments, the instructions, when executed by the processor, cause the processor to implement a second opinion recommendation system that operates to analyze electronic medical records (EMRs) of a patient to identify a medical condition associated with the patient; identify a set of treatments from a corpus of medical treatment guidelines for the patient's medical condition; evaluate the EMRs of the patient to identify a current treatment being performed to treat the patient's medical condition; compare the current treatment to the set of treatments identified from the corpus of medical treatment guidelines for the patient's medical condition; responsive to identifying a treatment in the set of treatments corresponding to the current treatment, determine differences between the identified treatment and the current treatment; and send a notification indicating the determined differences between the identified treatment and the current treatment and a recommendation that a second opinion should be sought.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example block diagram of a second opinion recommendation system in accordance with one illustrative embodiment.

FIG. 2 is an example diagram of a cognitive health system in which aspects of the present invention may be implemented;

FIG. 3 is a block diagram of an example data processing system in which aspects of the illustrative embodiments are implemented;

FIG. 4 is an example diagram illustrating an interaction of elements of a healthcare cognitive system in accordance with one illustrative embodiment; and

FIG. 5 is a flowchart outlining an example operation of a second opinion recommendation system in accordance with one illustrative embodiment

### DETAILED DESCRIPTION

The strengths of current cognitive systems, such as current medical diagnosis, patient health management, patient treatment recommendation systems, cognitive medical decision support systems, law enforcement investigation systems, and other decision support systems, are that they can provide insights that improve the decision making performed by human beings. For example, in the medical context, such cognitive systems may improve medical practitioners' diagnostic hypotheses, can help medical practitioners avoid missing important diagnoses, and can assist medical practitioners with determining appropriate treatments for specific diseases. However, current systems still suffer from significant drawbacks which should be addressed in order to make such systems more accurate and usable for a variety of applications as well as more representative of the way in which human beings make decisions, such as diagnosing and treating patients. In particular, one drawback of current systems is that, for patients with chronic medical conditions (e.g., hypertension, type 2 diabetes, osteoporosis, etc.), the patients take several medications at the same time. A patient often relies on one principle primary care physician (PCP) to manage his/her care given that the chronic medical condition is not too severe (no hospital admissions), trusting the PCP to be filly informed of all the treatments, medications, and the like, that the patient has undergone or is currently engaged in/taking. However, as physicians are required to spend less and less time with patients due to the increased number of patients that they must see, and additional record keeping and other duties they must engage in, as it becomes more likely that a physician may miss information in the patient's electronic medical record (EMR) indicating a treatment or medication. Therefore, the drawback of current systems is that there are no systems that actively monitor a patient's medical condition and propose a second opinion to the patient or physician when conditions warrant one (a patient may not even think about this possibility because he/she relies 100% on his/her PCP).

The illustrative embodiments provide mechanisms for evaluating a patient's electronic medical records (EMRs) to determine a potential need for a second opinion. As noted above, patients often rely on a single physician to provide their care and do not generally monitor the treatment for potential over treatment or under treatment, i.e. they trust their physician to know the treatments that are appropriate for their medical conditions. Thus, in some illustrative embodiments, the mechanisms may include a second opinion recommendation system that constantly observes the patient's EMR, including all medications, vitals (e.g., blood pressure), lab tests (e.g., A1C), etc., to determine whether the patient might be at a high risk to be over treated or under treated. Over treatment may include, hut is not limited to, the prescribing of unnecessary medications, treatments that are not leading to successful outcomes relative to other patients having a similar medical condition and other patient characteristics. Under treatment may include, but is not limited to, not receiving medication prescriptions or procedures that are recommended by treatment guidelines for patients with certain symptoms or characteristics. In this case the system would recommend the patient to seek a second opinion from the same or a different physician or a creditable on-line medical knowledge database. In addition, social networking information may be evaluated to determine whether the physician is trusted by other patients with whom the patient communicates as potential sources for second opinions.

In particular, the second opinion recommendation system continuously analyzes a patient's EMRs including, but not limited to, the patient's clinical values, lab results, lifestyle information, and the like, to determine whether the patient is taking unnecessary medications or is undergoing a treatment that is not resulting in recovery commensurate with other similar patients having similar characteristics or whether the patient is not receiving the treatments recommended by treatment guidelines. It should be appreciated that this analysis may identify duplicate medications, medications being taken for a condition that is also affected by other lifestyle information which may mitigate the need for the medication, and other combinations of characteristics which may be indicative of the treatments/medications prescribed to the patient being instances of over-treatment. The second opinion recommendation system may also find instances when simpler drug regimen may be used without changing treatment effectiveness.

If such a condition is detected to be present, the second opinion recommendation system may communicate with the patient, the patient's primary care physician (PCP), and/or the particular physician responsible for the potentially unnecessary procedures, medications, or the like, to inform them of the need for a second opinion for the patient's medication condition. When communicating with the patient, the second opinion recommendation system may suggest other physicians from which to obtain a second opinion, which may take into consideration the medical condition for which the second opinion is to be obtained, the patient's current insurance coverage characteristics, etc. Moreover, in communicating with the PCP or other physician, the second opinion recommendation system may suggest a modification to the patient's medication prescription and the reasons for the suggested modification as per the medical treatment guidelines.

In addition, or alternatively, the second opinion recommendation system may collect user demographic and medical profile data from the patient's EMR and search a central EMR system to identify a sample size of patients that are from a similar demographic profile and have a same or similar medical conditions. The demographic data may include, but not be limited to, age, gender, race, lifestyle information, employment condition, social relationship information, and the like.

The second opinion recommendation system may compare the patient's treatment plan and recovery progress information obtained from the patient EMR with the selected sample of patients. The second opinion recommendation system may determine whether the present patient is on a common pattern of recovery progress with others in the same profile, or even with others in the sample that are using a same or similar treatment plan. A determination may be made as to whether the present patient's recovery progress differs from other patients by more than a predetermined amount of progress and if so, a notice may be generated to the patient that the patient should seek a second opinion for further treatment, or a notice may be generated to the physician to reconsider the treatment for this patient.

In addition, or alternatively, the second opinion recommendation system may access the present patient's personal information management system, such as an electronic mail system, social networking websites utilized by the patient, or the like, to identify other users with which the patient communicates, as well as content of the communications exchanged. From the second opinion recommendation system a social graph may be generated that indicates links between the patient and other users, potentially based on the personal information for other users with which the present patient communicates, e.g., present patient communicates with patient B who sees doctor Z for treatment for a similar medical condition. From the social graph, potential sources of a second opinion may be identified, e.g., other users that the patient communicates directly with that are medical professionals, other users that have similar demographics and/or medical conditions with which the user communicates and who see a particular medical professional for treatment, etc. In this way, a set of one or more medical professionals that the present patient may find trustworthy may be identified and used as a basis for generating a notification and suggestion of where to obtain a second opinion for treatment of the present patient's medical condition.

Thus, the mechanisms of the illustrative embodiments may be integrated in, or operate with, a decision support system, which may be implemented as a cognitive system, which provides notifications and/or the recommendations based on evaluating the patient's EMR and comparing the obtained information to medical treatment guidelines, treatment other patients are receiving, etc.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to he limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may he utilized in addition to, or in replacement of, the examples provided herein without departing from the spirit and scope of the present invention.

As noted above, the present invention provides mechanisms for evaluating a patient's electronic medical records (EMRs) to determine a potential need for a second opinion. The illustrative embodiments of the present invention utilize automated specially configured computing systems that automatically analyze electronic medical records (EMRs) of a patient to identify a medical condition associated with the patient. Based on the identified medical condition, the computing systems identify a set of treatments from a corpus of medical treatment guidelines for the patient's medical condition. The computing systems then evaluate the electronic medical records (EMRs) of a patient to identify a current treatment being performed to treat the patient's medical condition. By comparing the current treatment to the set of treatments identified from the corpus of medical treatment guidelines for the patient's medical condition, the computing systems differences between the identified treatment and the current treatment, i.e. whether the patient is being treated properly, under treated, or over treated. The computing sends a notification indicating the determined differences between the identified treatment and the current treatment and a recommendation that a second opinion should be sought. In some cases, the results of the cognitive analysis may initiate automated communication with the medical professional, or with the patient themselves, to provide notifications of a potential need for a second opinion.

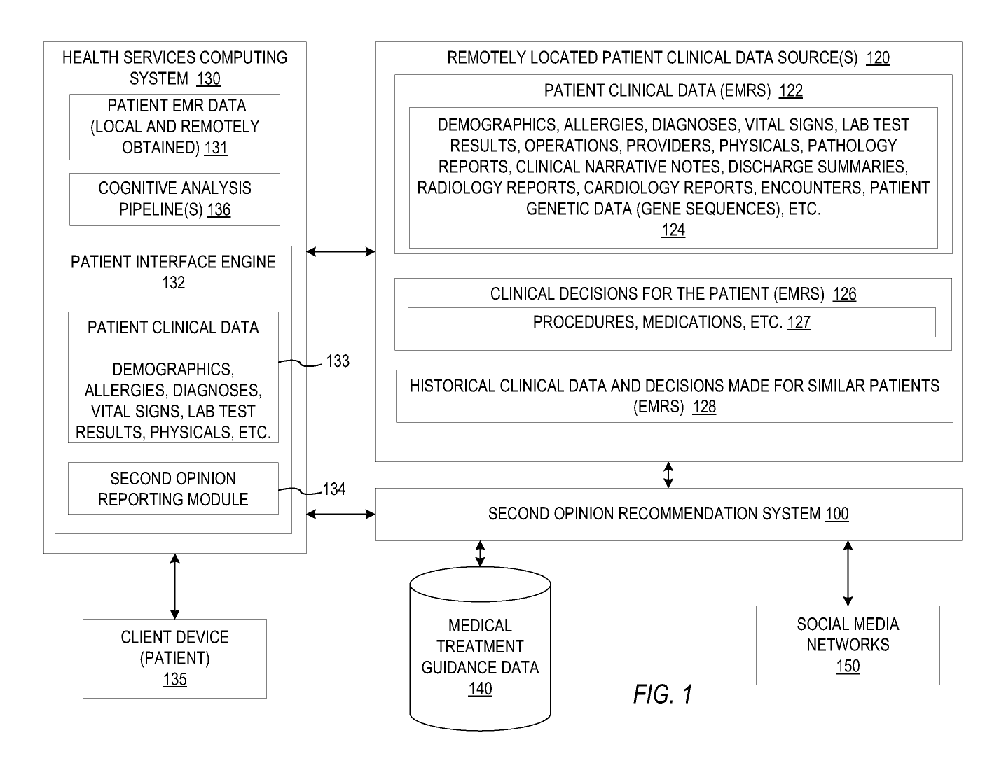

FIG. 1 is an example block diagram of a second opinion recommendation system in accordance with one illustrative embodiment. As shown in FIG. 1, second opinion recommendation system 100 may communicate with health services computing system 130, corpus of medical treatment guidance data 140, and social media networks 150. Second opinion recommendation system 100 may comprise health services computer system 130 interface logic, a network interface for communicating with the corpus of medical treatment guidance data 140 and social media networks 150, as well as other control logic for orchestrating the operations of second opinion recommendation system 100. This logic is not explicitly separately shown in FIG. 1, but is considered to be present in second opinion recommendation system 100 as well as other logic for performing functions or operations not specifically attributed to the second opinion recommendation system 100.

Health services computing system 130 may be any computing system associated with a provider of health services, such as a hospital, physician's office, pharmacy, clinic, laboratory, medical imaging facility, or the like. Health services computing system 130 may be itself a computer that a physician accesses to obtain information about the patient, or may be a server or other computing device that provides data to client computing device 135, such as portable tablet computing devices, workstations, or the like, that the physician may use to access the patient information. For example, healthcare services computing system 130 may he a server located in a healthcare facility, such as a hospital, physician's office, clinic, or the like, and the physician or other healthcare worker may utilize a client computer 135 in an exam room, a portable tablet computing device 135, or the like, that has a communication connection with health services computing system 130 to access the patient's information from the server for display on the client computer, e.g., the interface engine 132 which will be described hereafter. Moreover, in some cases client device 135 may in fact be a client device associated with the patient through which the patient may access interface engine 132 to obtain information about their own health, medical conditions, and treatments. Appropriate privacy protections, as are generally known in the art, may be employed to ensure that unauthorized access to personal patient information is not permitted.

Health services computing system 130 stores or has access to patient EMR data 131 that is specific to the healthcare services computing system 130 stored locally, and/or remotely located patient EMR and clinical data, such as may be accessed via a network interface and one or more data networks from a remotely located computing system, such as remotely located patient clinical data source(s) 120. Patient EMR data 131 may be obtained from remotely located patient clinical data sources 120 and/or may be stored locally. The patient EMR data 131 may comprise various types of patient clinical data and other patient information from a variety of different source computing systems associated with health service providers, medical product providers, pharmacies, insurance companies, and the like. For example, patient EMR data 131 may comprise a collection of clinical data for a patient obtained from hospitals, doctor offices, pharmacies, medical equipment supply companies, health insurance companies, clinics, medical imaging service providers, medical laboratories, etc. In some cases, patient clinical data may also be obtained from wearable or portable health and activity monitoring devices or applications executing on portable devices, e.g., FitBit™, applications executing on portable computing devices, smartphones, or the like. The locally stored patient EMR data 131 may be previously obtained from remotely located patient clinical data sources 120 and stored locally, or may be obtained locally from medical personnel input based on patient interactions, e.g., at a doctor's office. Local patient EMR data 131 may be input via local computing device interaction by medical personnel.

Remotely located patient clinical data sources 120 may be accessed via one or more data networks (not shown in FIG. 1) to obtain patient clinical data information, which may be collected from one or more other clinical data sources (not shown) and/or collected and stored from the patient's portable health monitoring device(s). That is, health services computing system 130 may access information stored in the remotely located patient clinical data source 120 via a network interface and provide the retrieved patient information, or a designated portion thereof to which the patient has granted access, to health service computing system 130.

In some illustrative embodiments, remotely located patient clinical data source 120 may be a cloud computing system comprising a plurality of computing device that share the responsibility for maintaining and protecting patient medical information, such as may be provided in one or more patient EMR data structures, obtained from one or more patient information source computing systems, such as via one or more data networks. Moreover, remotely located patient clinical data source 120 may obtain patient information collected from the patient's portable health monitoring device(s).

Remotely located patient clinical data source 120 may store a variety of different types of patient clinical data 122 obtained from a variety of different clinical data sources. For example, the patient clinical data 122 that may be collected and stored in the remotely located patient clinical data source 120 may include, for each of a plurality of patients, demographic information, allergy information, diagnosis information, vital sign information, laboratory test results information, medical procedure (operations) information, health services provider information and health insurance provider information, information regarding physical exams, pathology reports, clinical narrative notes, hospital/clinical discharge summary information, radiology reports, cardiology reports, other patient encounter information, patient genetic data (e.g., gene sequence data), or the like 124. Remotely located patient clinical data source 120 may also store clinical decisions for the patient 126, such as procedures, medication, or the like 127, as well as historical clinical data and decision made for similar patients 128, i.e. patients treating the same medical condition as that of the current patient.

Second opinion recommendation system 100 may cognitively process and analyze patient EMR data 131, either obtained locally or remotely from the remotely located patient clinical data source 120, in order to evaluate the patient EMR data and determining whether a second opinion should be sought. That is, second opinion recommendation system 100 analyzes patient EMR data 131 including, but not limited to, the patient's clinical values, lab results, lifestyle information, and the like, to determine whether the patient is taking unnecessary medications or is undergoing a treatment that is not resulting in recovery commensurate with other similar patients having similar characteristics or whether the patient is not receiving the treatments recommended by treatment guidelines. In order to perform this determination, second opinion recommendation system 100 analyzes the analyzes patient EMR data 131 that is specific to the healthcare services computing system 130 stored locally and/or remotely located patient EMR and clinical data to identify a medical condition associated with the patient as well as a current treatment being performed to treat the medical condition. For the current treatment being performed, second opinion recommendation system 100 identifies from patient EMR data 131 the current procedures, medications, or the like, that the patient is currently engaged in/taking.

Based on the identified medical condition, the second opinion recommendation system 100 identifies one or more medical treatments for treating the medical condition of the patient from the corpus of medical treatment guidance data 140. Additionally, based on the identified medical condition, second opinion recommendation system 100 analyzes the historical clinical data and decisions (EMRs) made for similar patients to identify one or more other patients that have a same or similar medical condition. The identification of the similar patients may be made simply on having the same medical condition or, in order to provide a smaller sample of similar patients, second opinion recommendation system 100 may also use demographic data associated with the patient to identify similar patients. That is, second opinion recommendation system 100 identifies user demographic and medical profile data from patient EMR data 131. Then using that user demographic and medical profile data, second opinion recommendation system 100 identifies similar patients that are from a similar demographic profile and have a same or similar medical conditions. The demographic data may include, but not be limited to, age, gender, race, lifestyle information, employment condition, social relationship information, and the like.

Second opinion recommendation system 100 then compares the patient's treatment plan and recovery progress information obtained from the patient EMR with the selected sample of similar patients and the one or more medical treatments for treating the medical condition of the patient. Second opinion recommendation system 100 may determine whether the present patient is on a common pattern of recovery progress with other patients with the similar demographic profile and/or or even with other patients using a same or similar treatment plan. A determination may be made as to whether the present patient's recovery progress differs from other patients by more than a predetermined amount of progress and if so, a notice may be generated to the patient that the patient should seek a second opinion for further treatment, or a notice may be generated to the physician to reconsider the treatment for this patient. That is, by comparing the patient's progress of the medical condition to other patients with the same medical condition, same demographic profile, and/or undergoing a same or similar treatment, second opinion recommendation system 100 may apply cognitive analysis logic, such as may he provided as part of one or more cognitive analysis pipelines 136, to the current procedures, medications, or the like, that the patient is currently engaged in/taking and to the current procedures, medications, or the like, that other patients with the same medical condition are currently engaged in/taking.

The cognitive analysis pipeline(s) 136 may comprise one or more cognitive analytics modules (not shown) that evaluate the received patient information (patient EMR data, clinical data, portable health monitor device captured information, and the like), previous medical conditions being treated, and the particular treatments prescribed, and may apply medical knowledge, treatment guidelines, and the like, that may be provided in electronic documents of a corpus or otherwise stored electronically for application to this patient information. The application of the knowledge from these medical resources to the patient information by the one or more cognitive analytics modules of the cognitive analysis pipeline(s) 136 provides an indication of the most relevant change information in the patient's medical condition and adherence to previously prescribed treatments. Moreover, in some illustrative embodiments, this change information may be used to drive further cognitive operations such that the cognitive analysis pipeline(s) 136 may generate treatment recommendations for the patient, recommended modifications to existing treatments, or even specific targeted warning messages, notifications, or the like, to be transmitted to the patient's client device or physician's client device 135.

Additionally or alternatively, second opinion recommendation system 100 may compare the progress of the patient's medical condition to the progress of other patients with the same medical condition that have undergone the current treatment the patient is currently engaged in/taking but have moved to another treatment to treat the medical condition. By applying the cognitive analysis logic to the current procedures, medications, or the like, that the patient is currently engaged in/taking and to the current procedures, medications, or the like, that other patients with the same medical condition are currently engaged in/taking, second opinion recommendation system 100 may identify a treatment that may work better for the patient.

Additionally or alternatively, second opinion recommendation system 100 also compares the current procedures, medications, or the like, that the patient is currently engaged in/taking to those one or more medical treatments for treating the medical condition of the patient identified from the corpus of medical treatment guidance data 140. That is, second opinion recommendation system 100 applies cognitive analysis to identify whether the current procedures, medications, or the like, that the patient is currently engaged in/taking fit within one or more of the medical treatments recommended for treating the medical condition.

Based on any or all of the comparisons, second opinion recommendation system 100 calculates a second opinion recommendation score to indicate differences between the identified treatment and the current treatment, i.e. whether the patient is at a place that is common in the recovery process, the patient is being under treated, or the patient is being over treated. A place that is common in the recovery process is identified as the patient being on a common pattern of recovery progress with others in the same profile, or even with others in the sample that are using a same or similar treatment plan. Over treatment may include, but is not limited to, the prescribing of unnecessary medications, treatments that are not leading to successful outcomes relative to other patients having a similar medical condition and other patient characteristics. Under treatment may include, but is not limited to, not receiving medication prescriptions or procedures that are recommended by treatment guidelines for patients with certain symptoms or characteristics. For example, second opinion recommendation system 100 may calculate the second opinion recommendation score in a range of 0 to 1, where a second opinion recommendation score closer to 0 indicates that the patient is being treated properly and a second opinion recommendation score closer to 1 indicates that the patient should seek a second opinion. Utilizing a predetermined threshold indicating that a second opinion should be sought, if the calculated second opinion recommendation score is above the predetermined threshold, second opinion recommendation system 100 would generate a recommendation for the patient to seek second opinion from the same or a different physician or a creditable on-line medical knowledge database.

If a recommendation is generated, second opinion recommendation system 100 may communicate with the patient, the patient's primary care physician (PCP), and/or the particular physician responsible for the potentially unnecessary procedures, medications, or the like, via second opinion reporting module 134, to inform them of the need for a second opinion for the patient's medication condition. When communicating with the patient, second opinion recommendation system 100 may suggest other physicians from which to obtain a second opinion, which may take into consideration the medical condition for which the second opinion is to be obtained, the patient's current insurance coverage characteristics, etc.

It should be appreciated that the cognitive analysis performed by second opinion recommendation system 100 may further identify duplicate medications, medications being taken for a condition that is also affected by other lifestyle information which may mitigate the need for the medication, and other combinations of characteristics which may be indicative of the treatments/medications prescribed to the patient being instances of over-treatment, Second opinion recommendation system 100 may also find instances when simpler drug regimen may be used without changing treatment effectiveness. Therefore, in communicating with the PCP or other physician via second opinion reporting module 134, second opinion recommendation system 100 may suggest a modification to the patient's medication prescription and the reasons for the suggested modification as per the medical treatment guidelines.

In addition or alternatively, second opinion recommendation system 100 may access the patient's personal information management system, such as an electronic mail system, social networking websites utilized by the patient, or the like, to identify other users with which the patient communicates, as well as content of the communications exchanged. From second opinion recommendation system 100 a social graph may be generated that indicates links between the patient and other users, potentially based on the personal information for other users with which the present patient communicates, e.g., present patient communicates with patient B who sees doctor Z for treatment for a similar medical condition. From the social graph, second opinion recommendation system 100 may identify potential sources of a second opinion, e.g., other users that the patient. communicates directly with that. are medical professionals, other users that have similar demographics and/or medical conditions with which the user communicates and who see a particular medical professional for treatment, etc. In this way, a set of one or more medical professionals that the present patient may find trustworthy may be identified and used as a basis for generating a notification and suggestion via second opinion reporting module 134 of where to obtain a second opinion for treatment of the present patient's medical condition.

The notifications, recommendations, or the like, generated by second opinion recommendation system 100 may be provided to interface engine 132 Which generates a interface in which the relevant clinical data of the patient 133 as well as second opinion reporting via second opinion reporting module 134, may be provided in an accentuated manner. In the depicted example, second opinion reporting module presents one or more of a recommendation to seek a second opinion, potentially unnecessary procedures, medications, or the like, other physicians from which to obtain a second opinion, as well as the patient's clinical data, i.e. current medical conditions, treatments, etc., which may be retrieved from the EMR data 131 and corresponding relevant information that is relevant to the medical conditions, treatment, or overall health of the patient.

As shown in FIG. 1, the results of the cognitive analysis of second opinion recommendation system 100 and/or cognitive analysis pipelines 136, as well as the clinical data and recommendation, notification, or the like, may be provided to the physician interface engine 132 which may construct a physician interface for output to a client device 135. The physician interface may comprise portions of a graphical user interface that set forth the relevant patient clinical data 133 as well as second opinion reporting module 134. The relevance of portions of the clinical data and the lifestyle behavior information may be evaluated by cognitive analysis pipelines 136 based on the particular medical conditions associated with the patient, the particular treatments associated with the patient, and the particular symptoms reported by the patient, based on medical knowledge obtained from medical knowledge resources (not shown), such as medical guidelines documents, physician desk references, pharmacology reference data, and the like, which may be provided in electronic form as part of a medical reference electronic corpus. Thus, in some illustrative embodiments, the physician interface generated by the physician interface engine 132 and provided to the client device 135 may comprise subsets of the clinical data obtained from the patient EMR data 131, and subsets of the lifestyle behavior information generated by second opinion recommendation system 100, that are relevant to the patient's medical condition, treatments, and reported symptoms. In some cases, the interface may further include subsets of such information that are generally applicable to all patients' overall health evaluation. Moreover, as noted above, the interface may present information relevant to both biological health as well as mental health.

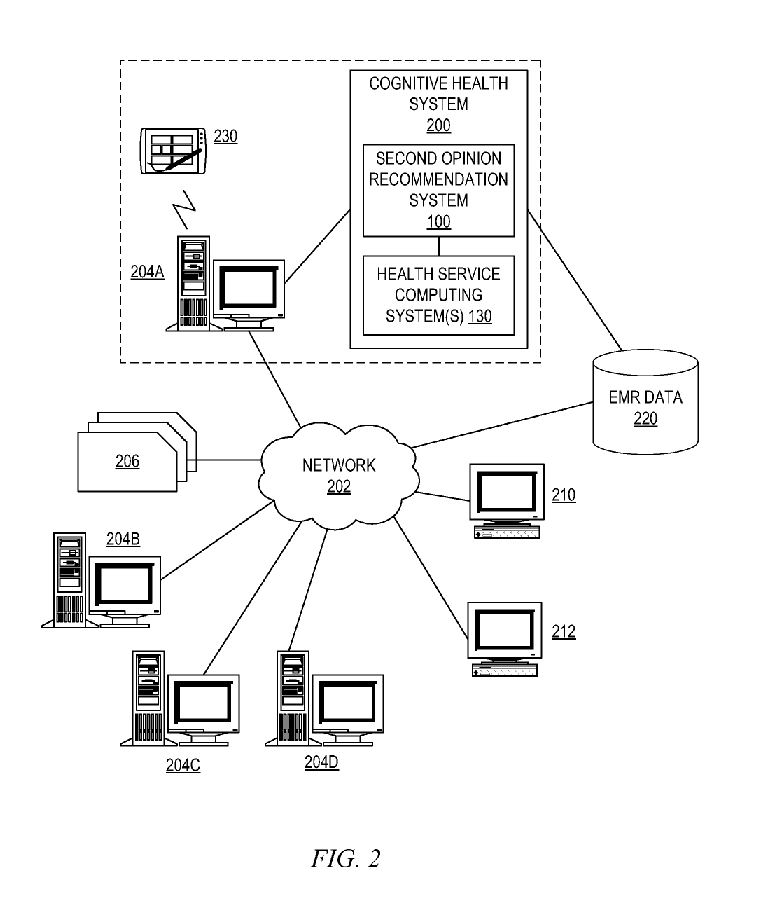

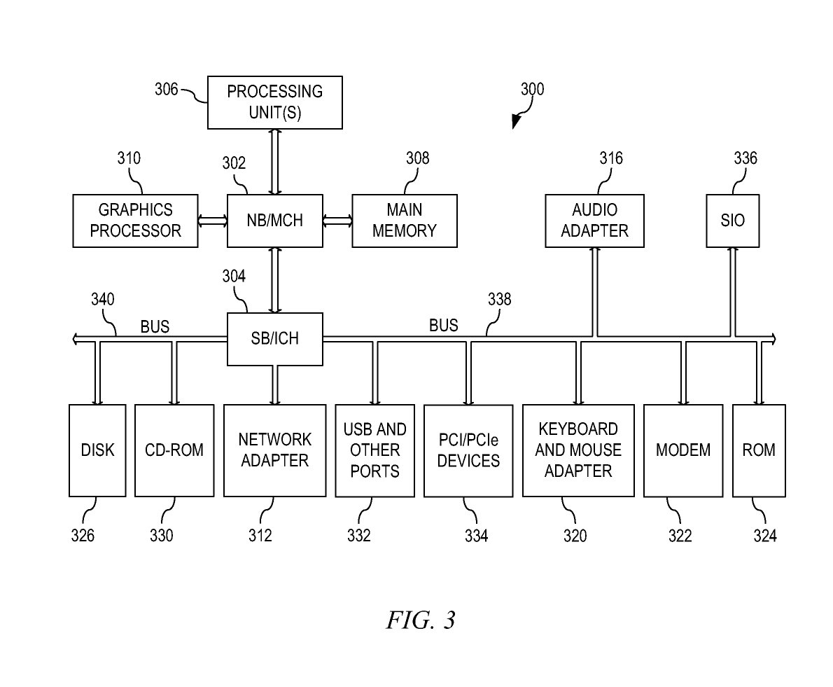

It should be appreciated that the illustrative embodiments may be utilized in many different types of data processing environments. In order to provide a context for the description of the specific elements and functionality of the illustrative embodiments, FIGS. 2-3 are provided hereafter as example environments in which aspects of the illustrative embodiments may be implemented. It should be appreciated that FIGS. 2-3 are only examples and are not intended to assert or imply any limitation with regard to the environments in which aspects or embodiments of the present invention may be implemented. Many modifications to the depicted environments may be made without departing from the spirit and scope of the present invention.

FIGS. 2-3 are directed to describing an example cognitive system for healthcare applications which implements a request processing apparatus, request processing methodology, and request processing computer program product with which the mechanisms of the illustrative embodiments are implemented. In particular, the cognitive system may comprise second opinion recommendation system 100 and health services computing system 130 which operate in conjunction to analyze patient EMR data from clinical data sources, EMR data of other patients with the same or similar medication condition, and one or more treatments for the medical condition, to determine a potential need for a second opinion. This functionality may be performed periodically, according to a predetermined schedule, or in response to the detection of particular events. For example, the functionality may be performed responsive to a request from a patient, the patient's primary care physician (PCP), and/or the particular physician responsible for the potentially unnecessary procedures, medications, or the like.

As shown in FIG. 2, the cognitive health system 200, comprising second opinion recommendation system 100 and health services computing system(s) 130, is implemented on one or more computing devices 204A-D (comprising one or more processors and one or more memories, and potentially any other computing device elements generally known in the art including buses, storage devices, communication interfaces, and the like) connected to the computer network 202. For purposes of illustration only, FIG. 2 depicts the cognitive health system 200 being implemented on computing device 204A only, but as noted above the cognitive health system 200 may be distributed across multiple computing devices, such as a plurality of computing devices 204A-D. The network 202 includes multiple computing devices 204A-D, which may operate as server computing devices, and client computing devices 210-212, in communication with each other and with other devices or components via one or more wired and/or wireless data communication links, where each communication link comprises one or more of wires, routers, switches, transmitters, receivers, or the like.

In some illustrative embodiments, the cognitive health system 200 and network 202 enable automatic extraction and cognitive analysis of patient EMR data from clinical data sources, EMR data of other patients with the same or similar medication condition, and one or more treatments for the medical condition to determine a potential need for a second opinion. As described previously, second opinion recommendation system 100 in combination with the health service computing system 130 may also operate on stored EMR data and clinical data for the patient 220, which may be locally stored or remotely stored on other computing systems or network attached data storage that is accessible by the cognitive health system 200, as well as medical knowledge or treatment guidance data obtained from medical knowledge resources, such as may be provided in one or more corpora 206. For example, the cognitive health system 200 may access a corpus or corpora of electronic documents 206 via the network 202, where portions of the corpus or corpora 206 may be provided on one or more server computing devices, network attached storage devices, in one or more databases, or other computing devices not explicitly shown in FIG. 2. The network 202 includes local network connections and remote connections in various embodiments, such that the cognitive health system 200 may operate in environments of any size, including local and global, the Internet.

The electronic documents of the one or more corpora 206 may include any file, text, article, or source of data for use in the cognitive health system 200 and may be provided in a structured or unstructured manner, e.g., natural language documents which may be processed using natural language processing to extract medical knowledge, treatment characteristics, and the like. For example, the electronic documents of the one or more corpora 206 may comprise medical knowledge bases, medical condition diagnosis knowledge sources, treatment guidelines, and the like, that specify various knowledge, criteria, and characteristics of medical conditions and treatments for such medical conditions. This information may be correlated with the medical conditions and treatments associated with the patient. This information may be used to determine a potential need for a second opinion, The patient EMR data from clinical data sources, EMR data of other patients with the same or similar medication condition, and one or more treatments for the medical condition may further be evaluated based on this knowledge obtained from the one or more corpora 206 to determine what criteria are met or not met, what certain ranges of values may represent, what portions of treatments are satisfied and whether the progression of the treatment is positively/negatively affecting to the patient's overall health and particular medical conditions. This information may be used to identify relevant changes in patient medical condition, patient lifestyle (e.g., habits), and treatment adherence that are to be accentuated in a physician interface, as discussed previously,

The physician interface may be provided to, or otherwise accessed, by a physician or other medical personnel via a computing device 230. In the depicted example, the client computing device is a portable tablet type computing device 230 which has graphical display capabilities used to provide a graphical user interface output of the interface. The output on the computing device 230 may comprise patient clinical data and notification, recommendations, or the like, as noted previously. The interface displays the information gathered and evaluated by the cognitive analytics modules of second opinion recommendation system 100, the relevant clinical data retrieved from patient EMRs, and/or the results of cognitive analysis by one or more of second opinion recommendation system 100 or cognitive analysis pipelines 136, to determine a potential need for a second opinion. Moreover, the interface may display the correlations of such information in a way as to explain the determined impact and interaction of the patient's current treatment with the clinical data and the patient's health, medical conditions, and adherence to treatments, as previously described above. It should be appreciated that second opinion recommendation system 100 and cognitive analysis pipelines 136 may apply thresholds, logic functions, and the like, to determine a degree or level of relevance or importance which may be used to identify an accentuation or particular representation of the information in the interface and thereby direct the attention of the patient, the patient's primary care physician (PCP), and/or the particular physician responsible for the potentially unnecessary procedures, medications, or the like, to this information. In this way, the most relevant clinical data and notification, recommendations, or the like is made apparent to the physician, medical personnel, or even patient, based on the most current knowledge of the patient's medical condition and treatment from the patient EMR data.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 3 is provided as an example of one type of data processing system in which aspects of the present invention may be implemented. Many other types of data processing systems may be likewise configured to specifically implement the mechanisms of the illustrative embodiments.

FIG. 3 is a block diagram of an example data processing system in which aspects of the illustrative embodiments are implemented. Data processing system 300 is an example of a computer, such as server 204 or client 210 in FIG. 2, in which computer usable code or instructions implementing the processes for illustrative embodiments of the present invention are located. In one illustrative embodiment, FIG. 3 represents a server computing device, such as a server 204, which implements a cognitive system 200 that includes the mechanisms of the illustrative embodiments described herein.

In the depicted example, data processing system 300 employs a hub architecture including North Bridge and Memory Controller Hub (NB/MCH) 302 and South Bridge and Input/Output (I/O) Controller Hub (SB/ICH) 304. Processing unit 306, main memory 308, and graphics processor 310 are connected to NB/MCH 302. Graphics processor 310 is connected to NB/MCH 302 through an accelerated graphics port (AGP).

In the depicted example, local area network (LAN) adapter 312 connects to SB/ICH 304. Audio adapter 316, keyboard and mouse adapter 320, modem 322, read only memory (ROM) 324, hard disk drive (HDD) 326, CD-ROM drive 330, universal serial bus (USB) ports and other communication ports 332, and PCI/PCIe devices 334 connect to SR/ICH 304 through bus 338 and bus 340. PCI/PCIe devices may include, for example, Ethernet adapters, add-in cards, and PC cards for notebook computers. PCI uses a card bus controller, while PCIe does not. ROM 324 may be, for example, a flash basic input/output system (BIOS).

HDD 326 and CD-ROM drive 330 connect to SB/ICH 304 through bus 340. HDD 326 and CD-ROM drive 330 may use, for example, an integrated drive electronics (IDE) or serial advanced technology attachment (SATA) interface. Super I/O (SIO) device 336 is connected to SB/ICH 304.

An operating system runs on processing unit 306. The operating system coordinates and provides control of various components within the data processing system 300 in FIG. 3. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 300.

As a server, data processing system 300 may be, for example, an IBM® eServer™ System p® computer system, running the Advanced Interactive Executive (AIX®) operating system or the LINUX® operating system. Data processing system 300 may be a symmetric multiprocessor (SMP) system including a plurality of processors in processing unit 306. Alternatively, a single processor system may be employed.

Instructions for the operating system, the object-oriented programming system, and applications or programs are located on storage devices, such as HDD 326, and are loaded into main memory 308 for execution by processing unit 306. The processes for illustrative embodiments of the present invention are performed by processing unit 306 using computer usable program code, which is located in a memory such as, for example, main memory 308, ROM 324, or in one or more peripheral devices 326 and 330, for example.

A bus system, such as bus 338 or bus 340 as shown in FIG. 3, is comprised of one or more buses. Of course, the bus system may be implemented using any type of communication fabric or architecture that provides for a transfer of data between different components or devices attached to the fabric or architecture. A communication unit, such as modem 322 or network adapter 312 of FIG. 3, includes one or more devices used to transmit and receive data. A memory may be, for example, main memory 308, ROM 324, or a cache such as found in NB/MCH 302 in FIG. 3.

Those of ordinary skill in the art will appreciate that the hardware depicted in FIGS. 2 and 3 may vary depending on the implementation. Other internal hardware or peripheral devices, such as flash memory, equivalent non-volatile memory, or optical disk drives and the like, may be used in addition to or in place of the hardware depicted in FIGS. 2 and 3. Also, the processes of the illustrative embodiments may be applied to a multiprocessor data processing system, other than the SMP system mentioned previously, without departing from the spirit and scope of the present invention.

Moreover, the data processing system 300 may take the form of any of a number of different data processing systems including client computing devices, server computing devices, a tablet computer, laptop computer, telephone or other communication device, a personal digital assistant (PDA), or the like. In some illustrative examples, data processing system 300 may be a portable computing device that is configured with flash memory to provide non-volatile memory for storing operating system files and/or user-generated data, for example. Essentially, data processing system 300 may be any known or later developed data processing system without architectural limitation.

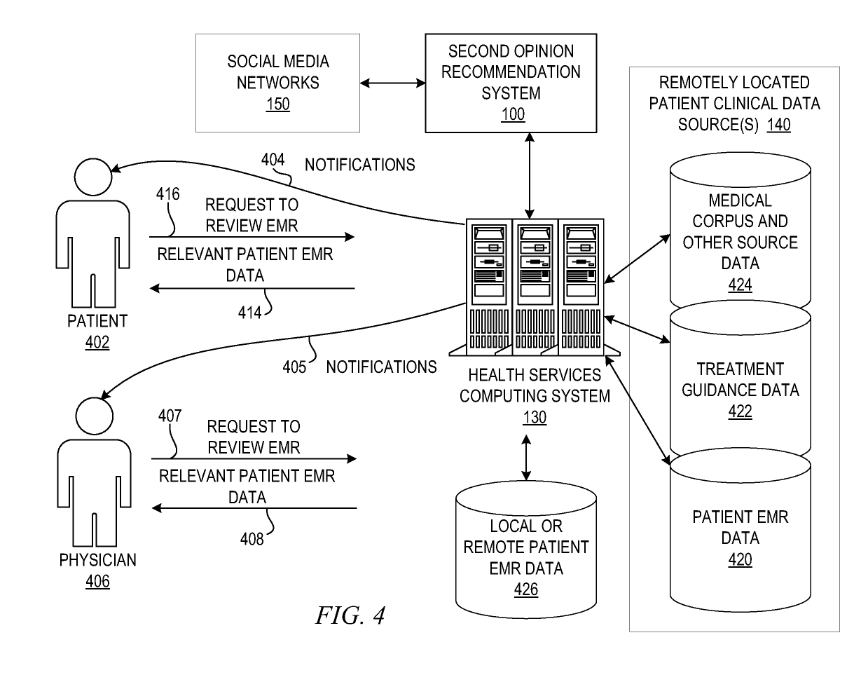

FIG. 4 is an example diagram illustrating an interaction of elements of a healthcare cognitive system in accordance with one illustrative embodiment. The example diagram of FIG. 4 depicts an implementation of a healthcare cognitive system that is configured to provide decision support services to a patient, the patient's primary care physician (PCP), and/or the particular physician responsible for the potentially unnecessary procedures, medications, or the like, in the way of cognitive evaluation of patient information collected from patient EMR data from clinical data sources, EMR data of other patients with the same or similar medication condition, and one or more treatments for the medical condition and the analysis of this information, as performed by second opinion recommendation system 100. The results of such evaluation may be presented to patient 402 and/or physician 406.

As shown in FIG. 4, second opinion recommendation system 100 may analyze a patient's EMR data, other patient's EMR data, medical guidelines, and social network information I order to determine whether a second opinion should be sought. This may be done automatically at periodic times, according to a schedule, or in response to a particular defined event. For example, in one illustrative embodiment, in response to physician 406 requesting a review of EMR data associated with a particular patient 402, the request 407 to review the EMR data may trigger the health services computing system 130 sending a request to second opinion recommendation system 100 to determine whether a second opinion should be sought with regard to a medical condition associated with patient 402. The request to review the patient 402 EMR data, i.e. request 407, may initiate a lookup of the patient EMR data (e.g., clinical data) in local patient EMR data storage 426, remotely located patient EMR data storage 420, or the like, and may identify a current medical condition for which the patient is being treated and the particular prescribed treatments, i.e. procedures, medications, or the like.

Based on the identified medical condition, the second opinion recommendation system 100 identifies one or more medical treatments for treating the medical condition of the patient gathered from the ingestion of medical corpus and other source data 424, treatment guidance data 422, and the like. Additionally, based on the identified medical condition, second opinion recommendation system 100 analyzes the historical clinical data and decisions (EMRs) made for similar patients in the remotely located patient EMR data storage 420 to identify one or more other patients that have a same or similar medical condition. The identification of the similar patients may be made simply on having the same medical condition or, in order to provide a smaller sample of similar patients, second opinion recommendation system 100 may also use demographic data associated with the patient to identify similar patients. That is, second opinion recommendation system 100 identifies user demographic and medical profile data from patient EMR data 426. Then using that user demographic and medical profile data, second opinion recommendation system 100 identifies similar patients that are from a similar demographic profile and have a same or similar medical conditions. The demographic data may include, but not be limited to, age, gender, race, lifestyle information, employment condition, social relationship information, and the like.

Second opinion recommendation system 100 then compares the patient's treatment plan and recovery progress information obtained from the patient's EMR data with the selected sample of similar patients and the one or more medical treatments for treating the medical condition of the patient. Second opinion recommendation system 100 may determine whether the present patient is on a common pattern of recovery progress with other patients with the similar demographic profile and/or or even with other patients using a same or similar treatment plan. A determination may be made as to whether the present patient's recovery progress differs from other patients by more than a predetermined amount of progress and if so, a notice may be generated to the patient that the patient should seek a second opinion for further treatment, or a notice may be generated to the physician to reconsider the treatment for this patient. That is, by comparing the patient's progress of the medical condition to other patients with the same medical condition, same demographic profile, and/or undergoing a same or similar treatment, second opinion recommendation system 100 may apply cognitive analysis logic to the current procedures, medications, or the like, that the patient is currently engaged in/taking and to the current procedures, medications, or the like, that other patients with the same medical condition are currently engaged in/taking.

Additionally or alternatively, second opinion recommendation system 100 may compare the progress of the patient's medical condition to the progress of other patients with the same medical condition that have undergone the current treatment the patient is currently engaged in/taking but have moved to another treatment to treat the medical condition. By applying the cognitive analysis logic to the current procedures, medications, or the like, that the patient is currently engaged in/taking and to the current procedures, medications, or the like, that other patients with the same medical condition are currently engaged in/taking, second opinion recommendation system 100 may identify a treatment that may work better for the patient.

Additionally or alternatively, second opinion recommendation system 100 may also compare the current procedures, medications, or the like, that the patient is currently engaged in/taking to those one or more medical treatments for treating the medical condition of the patient identified from the corpus of medical treatment guidance data 140. That is, second opinion recommendation system 100 applies cognitive analysis to identify whether the current procedures, medications, or the like, that the patient is currently engaged in/taking fit within one or more of the medical treatments recommended for treating the medical condition.

Based on any or all of the comparisons, second opinion recommendation system 100 calculates a second opinion recommendation score to indicate whether the patient is at a place that is common in the recovery process, the patient is being under treated, or the patient is being over treated. A place that is common in the recovery process is identified as the patient being on a common pattern of recovery progress with others in the same profile, or even with others in the sample that are using a same or similar treatment plan. Over treatment may include, but is not limited to, the prescribing of unnecessary medications, treatments that are not leading to successful outcomes relative to other patients having a similar medical condition and other patient characteristics. Under treatment may include, but is not limited to, not receiving medication prescriptions or procedures that are recommended by treatment guidelines for patients with certain symptoms or characteristics. For example, second opinion recommendation system 100 may calculate a second opinion recommendation score in a range of 0 to 1, where a second opinion recommendation score closer to 0 indicates that the patient is being treated properly and a second opinion recommendation score closer to 1 indicates that the patient should seek a second opinion. Utilizing a predetermined threshold indicating that a second opinion should be sought, if the calculated second opinion recommendation score is above the predetermined threshold, second opinion recommendation system 100 would generate a recommendation or notification 405 that a second opinion should be sought.

In another illustrative embodiment, as similar operation may be performed in response to patient 402 requesting a review of his/her EMR data, the request 416 to review the EMR data may trigger the health services computing system 130 sending a request to second opinion recommendation system 100 to determine whether a second opinion should be sought with regard to a medical condition associated with patient 402. The only difference would be that, if the calculated second opinion recommendation score is above the predetermined threshold, second opinion recommendation system 100 would generate a recommendation or notification 404 that a second opinion should be sought from the same or a different physician or a creditable on-line medical knowledge database.

Thus, if a recommendation notification 404, 405 is generated, second opinion recommendation system 100 may communicate with the patient, the patient's primary care physician (PCP), and/or the particular physician responsible for the potentially unnecessary procedures, medications, or the like, to inform them of the need for a second opinion for the patient's medication condition. When communicating with the patient, second opinion recommendation system 100 may suggest other physicians from which to obtain a second opinion, which may take into consideration the medical condition for which the second opinion is to be obtained, the patient's current insurance coverage characteristics, etc.

It should be appreciated that the cognitive analysis performed by second opinion recommendation system 100 may further identify duplicate medications, medications being taken for a condition that is also affected by other lifestyle information which may mitigate the need for the medication, and other combinations of characteristics which may be indicative of the treatments/medications prescribed to the patient being instances of over-treatment. Second opinion recommendation system 100 may also find instances when simpler drug regimen may he used without changing treatment effectiveness. Therefore, in communicating with the PCP or other physician, second opinion recommendation system 100 may suggest a modification to the patient's medication prescription and the reasons for the suggested modification as per the medical treatment guidelines.

In addition or alternatively, when suggesting a different physician, second opinion recommendation system 100 may access the patient's personal information management system, such as an electronic mail system, social networking websites utilized by the patient, or the like, to identify other users with which the patient communicates, as well as content of the communications exchanged. From second opinion recommendation system 100 a social graph may be generated that indicates links between the patient and other users, potentially based on the personal information for other users with which the present patient communicates, e.g., present patient communicates with patient B who sees doctor Z for treatment for a similar medical condition. From the social graph, second opinion recommendation system 100 may identify potential sources of a second opinion, e.g., other users that the patient communicates directly with that are medical professionals, other users that have similar demographics and/or medical conditions with which the user communicates and who see a particular medical professional for treatment, etc. In this way, a set of one or more medical professionals that the present patient may find trustworthy may be identified and used as a basis for generating a notification and suggestion of where to obtain a second opinion for treatment of the present patient's medical condition.

The results of the cognitive analysis performed by second opinion recommendation system 100 and health services computing system 130 may be utilized to generate notifications 404, 405 which may be sent to the patient and/or physician, or to generate an interface 408, 414 such as in the manner previously described above with regard to FIG. 1, for example. The resulting interface 408 setting forth relevant patient EMR data may be used by patient 402 or physician 406 so as to tailor the recommendation or notification to the most up-to-date understanding of the patient's needs as it contributes to the patient's overall health, specific medical conditions, and treatments.

That is, from the patient EMR data 420, 426, the medical conditions and the current treatments that patient 402 may be undergoing, may be identified. The corresponding information identified and/or generated second opinion recommendation system 100 based on the identifies information, that is relevant to the current treatments and/or medical conditions, may be identified and correlated with the treatments of patients with a same or similar medical condition undergoing or having undergone a same or similar treatment, The most up-to-date information may be reflected in a notification 404, 405 or interface 414, 408 presented to the patient 402 and/or physician 406 via the health services computing system 130 in association with the patient's EMR data when the patient 402 or physician 406 is viewing this information. This information may be correlated with information indicative of the important patient characteristics and health information for treating the medical condition(s), as may be obtained from medical guidelines, clinical guidance from subject matter experts, and the like, which may be stored as data structures in one or more resource data sources 422, 424. This information may then be used to select health information from the patient EMRs that are relevant to patient 402 which may then be identified in interface 414, 408 output to the patient 402 or physician 406 via the health services computing system 130.

It should be appreciated that while FIG. 4 depicts the patient 402 and physician 406 as human figures, the interactions with and between these entities may be performed using computing devices, medical equipment, and/or the like, such that entities 402 and 406 may in fact be computing devices, e.g., client computing devices. Interactions between the patient 402 or physician 406 and the health services computing system 130 will be electronic via a user computing device (not shown), such as a client computing device, portable computing device, or the like, communicating with the health services computing system 130 via one or more data communication links and potentially one or more data networks.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

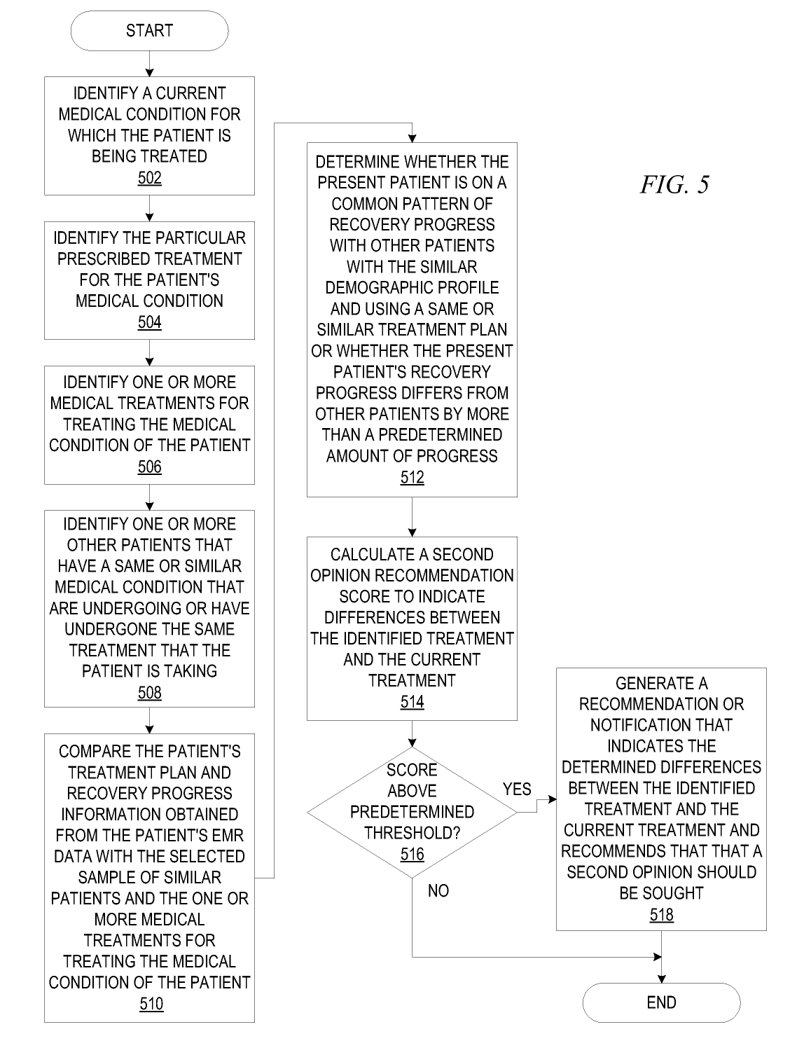

FIG. 5 is a flowchart outlining an example operation of a second opinion recommendation system in accordance with one illustrative embodiment. As shown in FIG. 5, the operation starts by the second opinion recommendation system identifying a current medical condition for which the patient is being treated (step 502) and identifying the particular prescribed treatment, i.e. procedures, medications, or the like, for the patient's medical condition (step 504). Based on the identified medical condition, the second opinion recommendation system identifies one or more medical treatments for treating the medical condition of the patient (step 506). Additionally, based on the identified medical condition, the second opinion recommendation system analyzes the historical clinical data and decisions (EMRs) made for similar patients to identify one or more other patients that have a same or similar medical condition that are undergoing or have undergone the same treatment that the patient is taking (step 508). The identification of the similar patients may be made simply on having the same medical condition or, in order to provide a smaller sample of similar patients, using demographic data associated with the patient to identify similar patients. That is, the second opinion recommendation system identifies user demographic and medical profile data from patient's EMR data. Then using that user demographic and medical profile data of the patient, the second opinion recommendation system identifies similar patients that are from a similar demographic profile and have a same or similar medical conditions. The demographic data may include, but not be limited to, age, gender, race, lifestyle information, employment condition, social relationship information, and the like.

The second opinion recommendation system then compares the patient's treatment plan and recovery progress information obtained from the patient's EMR data with the selected sample of similar patients and the one or more medical treatments for treating the medical condition of the patient (step 510). The second opinion recommendation system then determine whether the present patient is on a common pattern of recovery progress with other patients with the similar demographic profile and using a same or similar treatment plan or whether the present patient's recovery progress differs from other patients by more than a predetermined amount of progress (step 512). That is, by comparing the patient's progress of the medical condition to other patients with the same medical condition, same demographic profile, and/or undergoing or having undergone a same or similar treatment, the second opinion recommendation system may apply cognitive analysis logic to the current procedures, medications, or the like, that the patient is currently engaged in/taking and to the current procedures, medications, or the like, that other patients with the same medical condition are currently engaged in/taking.

Based on any or all of the comparisons, the second opinion recommendation system calculates a second opinion recommendation score to indicate differences between the identified treatment and the current treatment, i.e. whether the patient is at a place that is common in the recovery process, the patient is being under treated, or the patient is being over treated (step 514). A place that is common in the recovery process is identified as the patient being on a common pattern of recovery progress with others in the same profile, or even with others in the sample that are using a same or similar treatment plan. Over treatment may include, but is not limited to, the prescribing of unnecessary medications, treatments that are not leading to successful outcomes relative to other patients having a similar medical condition and other patient characteristics. Under treatment may include, but is not limited to, not receiving medication prescriptions or procedures that are recommended by treatment guidelines for patients with certain symptoms or characteristics. For example, the second opinion recommendation system may calculate a second opinion recommendation score in a range of 0 to 1, where a second opinion recommendation score closer to 0 indicates that the patient is being treated properly and a second opinion recommendation score closer to 1 indicates that the patient should seek a second opinion. The second opinion recommendation system determines whether the calculated second opinion recommendation score is above the predetermined threshold that indicates a second opinion should be sought (step 516). If at step 516 the calculated second opinion recommendation score is not above the predetermined threshold, then the operation terminates. However, if at step 516 the calculated second opinion recommendation score is above the threshold, the second opinion recommendation system generates a recommendation or notification that indicates the determined differences between the identified treatment and the current treatment and recommends that a second opinion should be sought (step 518).

Thus, if a recommendation or notification is generated, the second opinion recommendation system may send the recommendation or notification to the patient, the patient's primary care physician (PCP), and/or the particular physician responsible for the potentially unnecessary procedures, medications, or the like, to inform them of the need for a second opinion for the patient's medication condition (step 520). When communicating with the patient, second opinion recommendation system may suggest other physicians from which to obtain a second opinion, which may take into consideration the medical condition for which the second opinion is to be obtained, the patient's current insurance coverage characteristics, etc. The operation terminates thereafter.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for evaluating a patient's electronic medical records (EMRs) to determine a potential need for a second opinion. The mechanisms may include a second opinion recommendation system that observes the patient's EMR, including all medications, vitals (e.g., blood pressure), lab tests (e.g., A1C), etc., to determine whether the patient might be at a high risk to be over treated or under treated. Over treatment may include, but is not limited to, the prescribing of unnecessary medications, treatments that are not leading to successful outcomes relative to other patients having a similar medical condition and other patient characteristics. Under treatment may include, but is not limited to, not receiving medication prescriptions or procedures that are recommended by treatment guidelines for patients with certain symptoms or characteristics. In this case the system would recommend the patient to seek a second opinion from the same or a different physician or a creditable on-line medical knowledge database.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

1-20. (canceled)

21. A method, in a data processing system comprising at least one processor and at least one memory, the at least one memory comprising instructions executed by the at least one processor to cause the at least one processor to implement a second opinion recommendation system, wherein the second opinion recommendation system operates to:
analyzing, by the second opinion recommendation system, electronic medical records (EMRs) of a patient to identify a medical condition associated with the patient;
identifying, by the second opinion recommendation system, a set of treatments from a corpus of medical treatment guidelines for the patient's medical condition;
evaluating, by the second opinion recommendation system, the EMRs of the patient to identify a current treatment being performed to treat the patient's medical condition;
comparing, by the second opinion recommendation system, a recovery process of the patient to a recovery process of other patients that are undergoing a same treatment to that of the current treatment for the same or similar medical condition; and
responsive to the recovery process of the patient failing to coincide with the recovery process of the other patients that are undergoing the same treatment, sending, by the second opinion recommendation system, a notification indicating that a second opinion should be sought.

22. The method of claim 21, wherein the determined difference is one of being under treated or being over treated, wherein the patient being under treated is determined by the patient failing to receive medications or procedures that are recommended by treatment guidelines for patients with similar medical conditions, and wherein the patient being over treated is determined by the patient being prescribed medications or procedures that are not recommended by treatment guidelines for patients with similar medical conditions

23. The method of claim 21, further comprising:
comparing, by the second opinion recommendation system, a recovery process of the patient to a recovery process of other patients that have undergone a same treatment for the same or similar medical condition and have moved to other treatments for the medical condition; and
responsive to the recovery process of the patient failing to coincide with the recovery process of the other patients that have undergone the same treatment, sending, by the second opinion recommendation system, a notification indicating that the second opinion should be sought along with an indication of the other treatments for the medical condition.

24. The method of claim 23, wherein the other patients are further limited by having a similar demographic profile to that of the patient.

25. The method of claim 21, further comprising:
recommending, by the second opinion recommendation system, that a medical professional implementing the current treatment be consulted.

26. The method of claim 21, further comprising:
recommending, by the second opinion recommendation system, that a different medical professional other than the medical professional implementing the current treatment be consulted.

27. The method of claim 6, wherein the different medical professional is identified by analyzing social media of the patient.

28. A computer program product comprising a computer readable storage medium having a computer readable program stored therein, wherein the computer readable program, when executed on a computing device, causes the computing device to implement a second opinion recommendation system which operates to:
analyze electronic medical records (EMRs) of a patient to identify a medical condition associated with the patient;
identify a set of treatments from a corpus of medical treatment guidelines for the patient's medical condition;
evaluate the EMRs of the patient to identify a current treatment being performed to treat the patient's medical condition;
compare a recovery process of the patient to a recovery process of other patients that are undergoing a same treatment to that of the current treatment for the same or similar medical condition; and
responsive to the recovery process of the patient failing to coincide with the recovery process of the other patients that are undergoing the same treatment, send a notification indicating that a second opinion should be sought.

29. The computer program product of claim 28, wherein the determined difference is one of being under treated or being over treated, wherein the patient being under treated is determined by the patient failing to receive medications or procedures that are recommended by treatment guidelines for patients with similar medical conditions, and wherein the patient being over treated is determined by the patient being prescribed medications or procedures that are not recommended by treatment guidelines for patients with similar medical conditions

30. The computer program product of claim 28, wherein the computer readable program further causes the computing device to implement the second opinion recommendation system which operates to:
compare a recovery process of the patient to a recovery process of other patients that have undergone a same treatment for the same or similar medical condition and have moved to other treatments for the medical condition; and
responsive to the recovery process of the patient failing to coincide with the recovery process of the other patients that have undergone the same treatment, send a notification indicating that the second opinion should be sought along with an indication of the other treatments for the medical condition.

31. The computer program product of claim 30, wherein the other patients are further limited by having a similar demographic profile to that of the patient.

32. The computer program product of claim 28, wherein the computer readable program further causes the computing device to implement the second opinion recommendation system which operates to:
recommend that a medical professional implementing the current treatment be consulted.

33. The computer program product of claim 28, wherein the computer readable program further causes the computing device to implement the second opinion recommendation system which operates to:
recommend that a different medical professional other than the medical professional implementing the current treatment be consulted.

34. The computer program product of claim 33, wherein the different medical professional is identified by analyzing social media of the patient.

35. An apparatus comprising:
a processor; and
a memory coupled to the processor, wherein the memory comprises instructions which, when executed by the processor, cause the processor to implement a cognitive medical decision support system that operates to:
analyze electronic medical records (EMRs) of a patient to identify a medical condition associated with the patient;
identify a set of treatments from a corpus of medical treatment guidelines for the patient's medical condition;
evaluate the EMRs of the patient to identify a current treatment being performed to treat the patient's medical condition;
compare a recovery process of the patient to a recovery process of other patients that are undergoing a same treatment to that of the current treatment for the same or similar medical condition; and
responsive to the recovery process of the patient failing to coincide with the recovery process of the other patients that are undergoing the same treatment, send a notification indicating that a second opinion should be sought.

36. The apparatus of claim 35, wherein the determined difference is one of being under treated or being over treated, wherein the patient being under treated is determined by the patient failing to receive medications or procedures that are recommended by treatment guidelines for patients with similar medical conditions, and wherein the patient being over treated is determined by the patient being prescribed medications or procedures that are not recommended by treatment guidelines for patients with similar medical conditions

37. The apparatus of claim 35, wherein the instructions further cause the computing device to implement the second opinion recommendation system which operates to:
compare a recovery process of the patient to a recovery process of other patients that have undergone a same treatment for the same or similar medical condition and have moved to other treatments for the medical condition; and
responsive to the recovery process of the patient failing to coincide with the recovery process of the other patients that have undergone the same treatment, send a notification indicating that the second opinion should be sought along with an indication of the other treatments for the medical condition.

38. The apparatus of claim 37, wherein the other patients are further limited by having a similar demographic profile to that of the patient.

39. The apparatus of claim 35, wherein the instructions further cause the processor to implement the second opinion recommendation system which operates to:
recommend that a medical professional implementing the current treatment be consulted.

40. The apparatus of claim 35, wherein the instructions further cause the processor to implement the second opinion recommendation system which operates to:
recommend that a different medical professional other than the medical professional implementing the current treatment be consulted, wherein the different medical professional is identified by analyzing social media of the patient.
